(19) United States
(12) Patent Application Publication
(10) Pub. No.: US 2022/0339180 A1
(43) Pub. Date: Oct. 27, 2022

(54) Methods And Compositions For Treating Viral Diseases Using Combination Of Drugs

(71) Applicant: NATIONAL INSTITUTE OF IMMUNOLOGY, New Delhi (IN)

(72) Inventors: Tanmay MAJUMDAR, New Delhi (IN); Bichitra K. Biswal, New Delhi (IN); Prafullakumar Tailor, New Delhi (IN)

(21) Appl. No.: 17/241,130

(22) Filed: Apr. 27, 2021

(51) Int. Cl. A61K 31/7076; A61K 31/7056; A61K 31/708; A61K 31/05; A61K 31/7064; A61K 31/7072; A61K 31/404; A61K 31/44; A61P 31/14

(52) U.S. Cl. CPC A61K 31/7076; A61K 31/7056; A61K 31/708; A61K 31/05; A61K 31/7064; A61K 31/7072; A61K 31/404; A61K 31/44; A61P 31/14

(57) ABSTRACT

The present invention describes methods and compositions for treating viral diseases through novel combinational therapy of one or more anti-viral and anti-kinase drugs. The composition and method of the present invention are aimed at targeting a NiRAN domain of RNA dependent RNA polymerase (RdRp) as RdRp is key enzyme for virus replication of the viral pathogens. The compositions of the present invention comprise one or more kinase inhibitors in combination with one or more anti-viral agents. The combination therapy of these agents was found to exhibit synergistic effecta in inhibiting viral activity against several lethal viruses including but not limited to SARS-CoV-2.

### FIELD OF THE INVENTION

The present invention relates to methods and compositions for treating viral infections using a novel combinational therapy. In particular, the present invention provides methods and compositions comprising one or more anti-viral drug in combination with one or more anti-kinase drugs for treatment of viral diseases, particularly SARS-CoV-2 infection,

### BACKGROUND OF INVENTION

Repurposing known therapeutic agents for augmenting the efficacy and potency of known anti-viral drugs is an important strategy for treatment of viral diseases. This approach may serve as an effective tool in providing efficient and effective results at affordable cost.

Machitani et al. (2020) reviews the enzymatic function of RdRP in virus proliferation and tumor development. A recent outbreak of coronavirus disease (COVID-19) caused by the novel severe acute respiratory syndrome coronavirus 2 has driven a global pandemic with catastrophic consequences. The rapid development of promising therapeutic strategies against COVID-19 is keenly anticipated. WHO COVID-19 Weekly Epidemiological Report (22 Dec. 2020) reports that SARS-CoV-2, the causative agent of the on-going global pandemic COVID-19, is a recently emerged pathogen that has infected over 75 million and caused over 1.6 million deaths as of 20Dec. 2020.

Riou, J., and Althaus, C. L. (2020) reports that with a basic reproduction number ranging from 1.4 to 3.9, the disease quickly spread across the globe within past five months. The situation demands urgent attention from researchers worldwide in order to develop a better understanding of the viral pathogenesis, clinical presentations and biology of the disease. The understanding of disease will help to develop therapeutics such as several small molecule inhibitors targeting the viral proteins. Andersen et al. (2020) discusses that prevalent hypotheses suggest that multiple genome level recombination and zoonotic events between coronaviruses affecting human and bat host have resulted in the evolution of SARS-CoV-2.

However, it is worth noticing that the RNA dependent RNA polymerase (RdRp) molecule of SARS-CoV-2 remains largely unchanged at the protein sequence level when compared to previous human coronaviruses such as SARS-CoV, MERS-CoV, SARS-Hkul as well as non-human coronaviruses like Bat-CoV-Hku4 and Bat-CoV-ZC45, to name a few (Bell et. al., 2020). Despite being a well-known pathogen, following the outbreak of SARS-CoV in 2002, the functional aspects of the RdRp molecule from coronaviruses remains unclear. In addition, even limited information is available on the peculiar NiRAN and interface domains that are specific to the RdRp molecules of viruses of the order Nidovirales. The first ever study that defines the NiRAN domain from the RdRp/Nsp9 of equine arteritis virus (EAV), demonstrates a manganese-dependent covalent binding of guanosine phosphate and uridine phosphate to an invariant lysine residue from the NiRAN domain. The study proposes three possible molecular functions of the NiRAN domain: as a ligase, as a G′1P dependent 5′ nucleotidyltransferase and as a UTP dependent protein priming function facilitating the initiation of RNA replication (Lehmann et. al., 2015 and Posthma et. al., 2017).

Various studies from other RNA viruses have demonstrated the nucleotidyltransferase activities exhibited by the N-terminal regions of the respective RdRp molecules. However, these transferase activities have been attributed to both 5′-priming as well as terminal ribonucleotide addition functions. Moreover, RdRp enzymes are known to exhibit either a primer dependent or a primer independent initiation of RNA replication. Interestingly, nidovirus RdRp molecules have been experimentally demonstrated to possess both modes of initiation, thus hinting as a probable UTP mediated priming function of the NiRAN domain. An earlier cryo-EM structure of replicase polyprotein complex from SARS-CoV had significant regions of the NiRAN domain missing, thus failed to provide any functional association to this domain (Kirchdoerfer et. al., 2019). However, a recent study reporting the cryo-EM structure of the replicase polyprotein complex from SARS-CoV-2 provides the complete structural preview of the NiRAN domain (Gao et. al., 2020). Given the unavailability of SARS-CoV-2 specific therapies and rapid emergence of newer strains, drug repurposing might prove crucial in combating the on-going epidemic while simultaneously being cost and time effective.

Thus, NiRAN domain may prove an important target for inhibiting viral activity and developing anti-viral therapies against viruses harbouring this domain. The present invention thus provides novel combination therapy of known drugs/kinase inhibitors for augmenting antiviral efficacy of said drugs, by targeting the activity of NiRAN domain.

### OBJECTS OF THE INVENTION

An important object of the present invention is to provide methods of treating viral infections in mammals, including humans, by administering a combination of one or more anti-viral and/or anti-kinase drugs or pharmaceutically acceptable derivatives thereof using in silico analysis, biochemical activity, and in vitro infectious disease model.

Another important objective of the present invention is to provide a composition/formulation comprising one or more anti-viral and/or anti-kinase drugs or pharmaceutically acceptable derivatives thereof.

Still another objective of the present invention is to provide a method of treating viral disease(s) by administering one or more anti-viral and/or anti-kinase drugs or pharmaceutically acceptable derivatives thereof inhibiting the activity of NiRAN domain of RNA dependent RNA polymerase (RdRp).

Yet another objective of the present invention is to provide a dosage form comprising antiviral and kinase inhibitor in the variable range in a sequential/simultaneous manner.

A further objective of the present invention is to provide a combination therapy for the treatment of SARS-CoV-2 by targeting NiRAN domain of SARS-CoV-2 RNA dependent RNA polymerase (RdRp).

### SUMMARY OF THE INVENTION

The present invention provides methods and compositions for treating viral diseases through combinational therapy of one or more anti-viral and anti-kinase drugs. The composition and methods of the present invention are aimed at targeting a NiRAN domain of RNA dependent RNA polymerase (RdRp). RdRp is a key enzyme for virus replication of the viral pathogens. The compositions of the present invention comprise one or more kinase inhibitors in combination with one or more anti-viral agents. The combination therapy of these agents has been found to exhibit synergistic effect in inhibiting viral activity against several lethal viruses including but not limited to SARS-CoV-2. Thus, compositions and methods of the present invention provide an effective treatment strategy to prevent the viral multiplication and any emerging infection resulting from the same.

BRIEF DESCRIPTION OF THE FIGURES/ DRAWINGS

The embodiments of the disclosure itself, as well as a preferred mode of use, further objectives and advantages thereof, will best be understood by reference to the following detailed description of an illustrative embodiment when read in conjunction with the accompanying drawings as listed below:

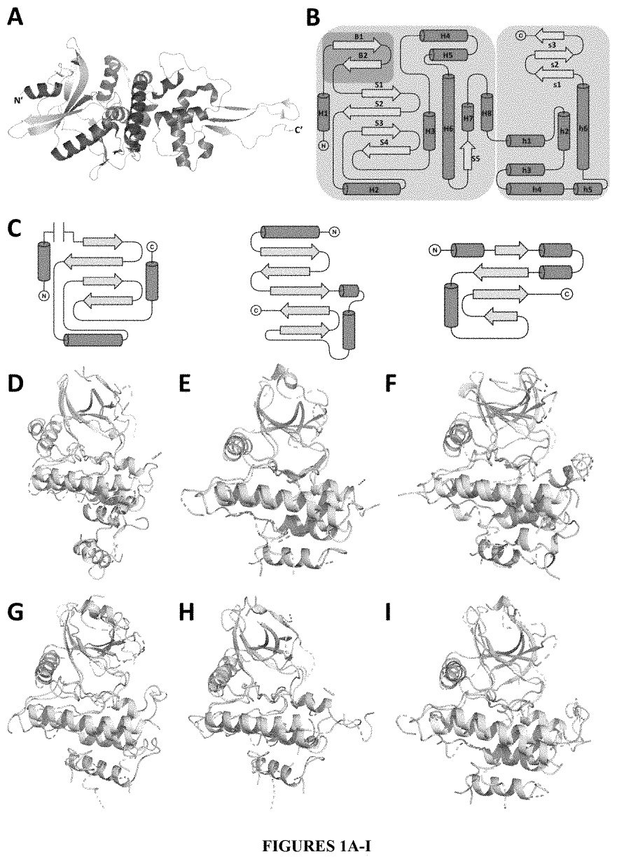

FIGS. 1A-I: The SARS-CoV-2-RdRp NiRAN domain present a kinase/phosphotransferase like structural organisation. (A) The NiRAN and the interface domains of the SARS-CoV-2-RdRp present an arrow-head like structure (helices in royal blue, strands in peach and loops in green). (B) The overall topology of the NiRAN (brown background, helices as marked as H and strand are marked as S) and interface domain (grey background, helices as marked as h and strand are marked as s) along with β-hairpin structure (brick red background, strand are marked as B). (C) The NiRAN domain possess a topology (left panel) that borrows elements from the canonical kinase fold (centre panel) as well as the non-canonical kinase fold of TgBPK1 (right panel). (D-I) The structural superimpositions of the secondary structural elements of CoV-2-RdRp NiRAN with known kinases reveal significant alignment in the antiparallel β-sheet and in the helices that follow. (D) Lim 2 kinase domain. (E) Syk kinase domain (F) O-mannosyl kinase domain. (G) IRAK4 kinase domain. (H) FGFR2 kinase domain. (I) Insulin receptor kinase domain. (The aforementioned kinases' structural elements are shown in cyan, CoV-2-RdRp NiRAN structural elements shown in yellow).

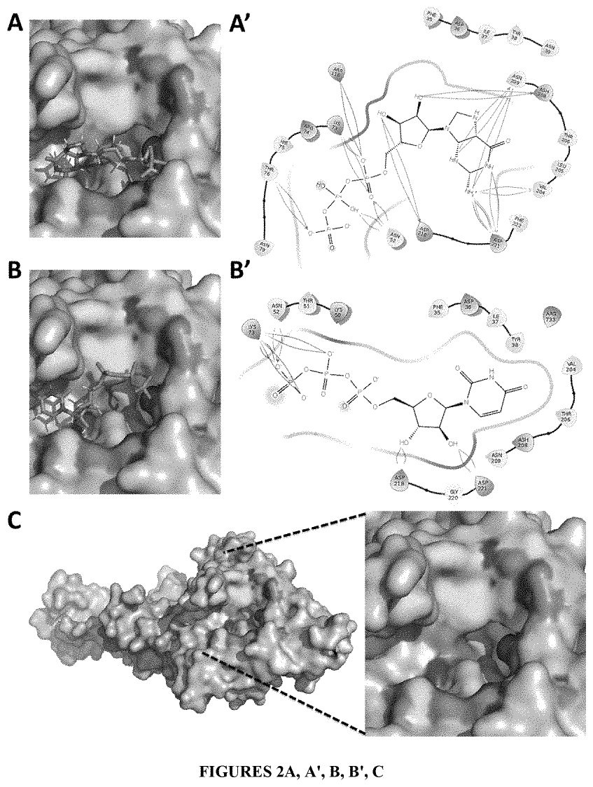

FIGS. 2A, A′, B, B′, C: The active site of SARS-CoV-2-RdRp NiRAN domain binds GTP and UTP and exhibits kinase like motifs. GTP and UTP bind at the probable active site of the NiRAN domain with notably low free binding energies. (A) GTP within the active site pocket. (A′) GTP binding reveals salt bridge interaction with K73 and H-bonding with Asp 116. (B) UTP within the active site pocket. (B′) UTP binding exhibits salt bridge interaction with K73. (C) The predicted active site of the NiRAN domain possess both charged and uncharged residues, where in the charged residues primarily line the entry points of the pocket and the uncharged residues line in the deeper sections. (Red indicates positively charged regions, blue indicates negatively charged regions and green indicates neutral regions, grey indicates regions beyond GTP-binding pocket).

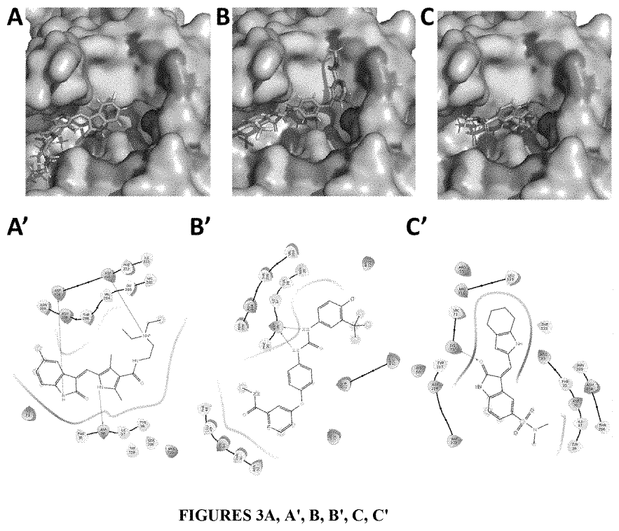

FIGS. 3A, A′, B, B′, C, C′: The active site of SARS-CoV-2-RdRp NiRAN domain binds kinase inhibitors. (A, B, C) Broad specificity kinase inhibitors bind within the predicted NiRAN active site with significantly low free binding energies. (Red indicates positively charged regions, blue indicates negatively charged regions and green indicates neutral regions, grey indicates regions beyond GTP-binding pocket): (A) Sunitinib within the active site pocket; (B) Sorafenib within the active site pocket; and (C) SU6656 within the active site pocket. The kinases inhibitors demonstrate H-bond interactions between with the enzymatically critical aspartate and lysine residues lining the active site- (A′) Sunitinib; (B′) Sorafenib; and (C′) SU6656.

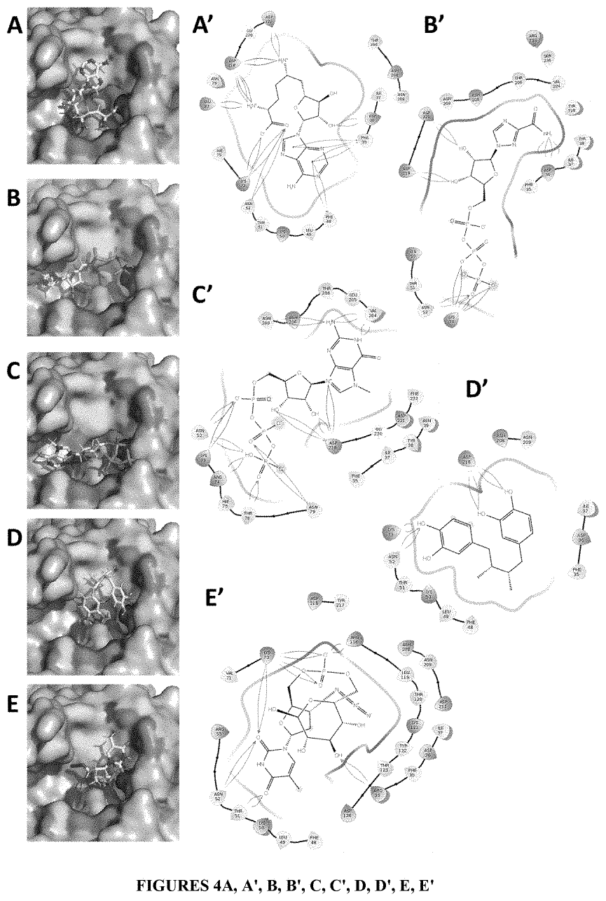

FIGS. 4A, A′, B, B′, C, C′, D, D′, E, E′: The binding and molecular interaction of the best five predicted nucleotidyltransferase inhibitors at the active site of CoV-2-RdRp NiRAN. The computationally directed binding of- (A) 65482; (B) 122108; (C) 135659024; (D) 4534; and (E) 23673624; within the active site pocket (Red indicates positively charged regions, blue indicates negatively charged regions and green indicates neutral regions, grey indicates regions beyond GTP-binding pocket). The molecular interactions between the inhibitors and the active site pocket reveal H-bonds, salt bridges and pi-pi interactions- (A′) 65482; (B′) 122108; (C′) 135659024; (D′) 4534; (E′) 23673624. Of note, the best predicted inhibitor- 65482 presents all the aforementioned molecular interactions.

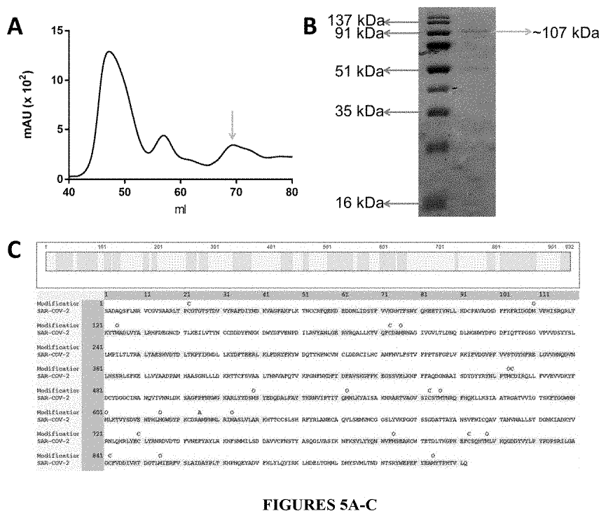

FIGS. 5A-C: Purification of SARS-CoV2-RdRp. (A) Size exclusion chromatogram of recombinant SARS-CoV-2 RdRp (The peak indicated with a blue arrow represents the purified protein sample). (B) SDS-PAGE profile of the purified SARS-CoV-2 RdRp (The band indicated with a blue arrow represents the purified protein sample and the molecular weight markers are indicated in red arrows).

FIGS. 6A-D: SARS-CoV-2 RdRp exhibits a kinase like activity. (A) The SARS-CoV-2 RdRp exhibits a kinase like activity akin to that of purified human Akt2. The ATP binding protein Bovine Senim albumin serves as a negative control (Data points show mean and standard error. The connecting curves represent non-linear regressions). (B) While treatment with all the kinase inhibitors abrogate the kinase like activity of SARS-CoV-2 RdRp, the activity human Akt2 is majorly inhibited by Sorafernib and SU6656 with Sunitinib only exhibiting mild inhibition. (C) Treatment of SARS-CoV2-RdRp with nucleotidyltransferase inhibitors135659024, 122108 and 65482 exhibit conspicuous inhibition of the kinase activity in micromolar concentrations. The compound 4534 however fails to exhibit any inhibitory potential (The bars represent mean and standard error. The symbols “*” and “**” represent significance for p-value less than 0.05 and 0.005, respectively. The symbol “ns” represents non-significance).(D)Treatment of SARS-CoV-2 infected epithelial cells with Sorafenib improves cell survival at concentrations similar to Remdesivir (A represents Sorafenib, R represents Remdesivir, concentrations shown for both compounds are 1, 5, 25 and 50 μM). The figures depict embodiments of the disclosure for purposes of illustration only.

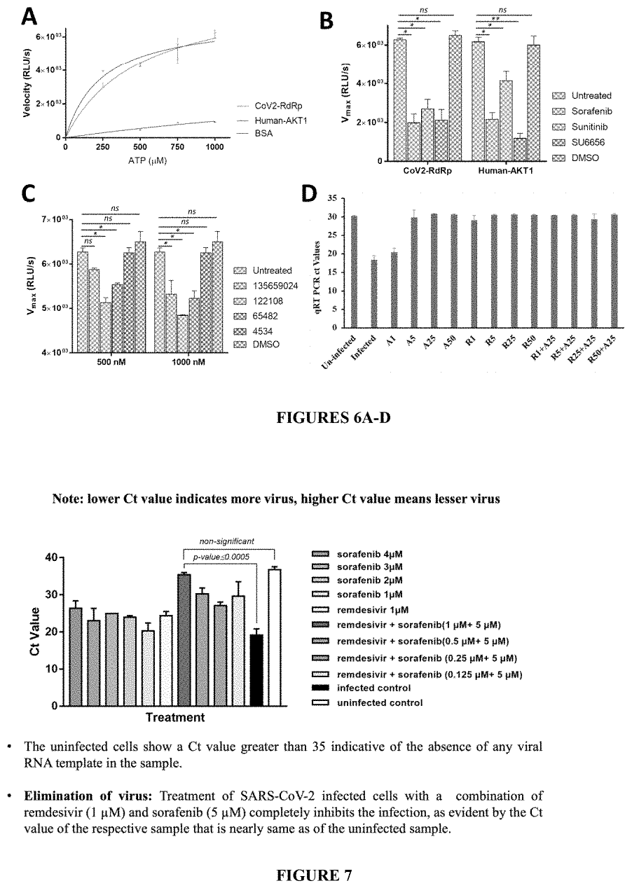

FIG. 7: Elimination of virus from infected cells. Cells were infected with SARS-CoV-2 and sorafenib and remdesivir were used in variable concentration to check combinatorial antiviral efficacy to clear the virus. Sorafenib (4 μM) or remdesivir (1μM) alone have effect to clear the virus. Nonetheless, in combination with sorafenib (5 μM) and remdesivir (1 μM) can eliminate the virus significantly. Due to clearance of virus from infected cells by sorafenib (5 μM) and remdesivir (1 μM), the Ct value of those treated cells is almost equal to uninfected cells. It proves that, combinatorial effect of sorafenib and remdesivir has a significant antiviral effect to eliminate SARS-CoV-2 infection.

### DETAILED DESCRIPTION OF THE INVENTION

The foregoing has broadly outlined the features and technical advantages of the present disclosure in order that the detailed description of the disclosure that follows may be better understood. It should be appreciated by those skilled in the art that the conception and specific embodiment disclosed may be readily utilized as a basis for modifying or designing other structures for carrying out the same purposes of the present disclosure.

The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### ABBREVIATIONS USED

SARS-CoV-2: Severe Acute Respiratory Syndrome Coronavirus-2
RdRp:RNA dependent RNA polymerase
NiRAN: Nidovirus RdRp associated nucleotidyltransferase domain
COVID-19: Coronavirus Disease 2019
EAV: Equine Arterivirus
ATP: Adenosine triphosphate
ADP: Adenosine diphosphate
GTP: Guanosine triphosphate
UTP: Uridine triphosphate

The present invention provides methods and compositions for treating viral diseases through combinational therapy of one or more anti-viral and anti-kinase drugs or a pharmaceutically acceptable derivative thereof The methods and compositions of the present invention are aimed at targeting NiRAN domain of RNA dependent RNA polymerase (RdRp) as RdRp is key enzyme for virus replication of the viral pathogens. The compositions of the present invention comprise one or more kinase inhibitors in combination with one or more anti-viral agents.

### EXAMPLES

1.1 Structural Identification of the NiRAN domain of RNA dependent RNA polymerase (RdRp) and its kinase like activity

The NiRAN and the interface domains span over residues 1-365 of the SARS-CoV-2 RdRp polypeptide sequence. These two domains form an arrow-head like structure, which acts as a base for the RdRp region of protein (FIG. 1A).). The overall topology suggests that the two distinct domains are connected by a small linker of 1 residue (FIG. 1B). A multiple sequence alignment of 17 RdRp polypeptide sequences from coronaviruses across multiple host species (human, bat, cow, pigs and rodents) revealed an absolute conservation of the residues- K73, R116, T123, D126, D218 and F219. A visualization of the conserved residues of the NiRAN domain shows that a significant majority of these residues are localized between the 4 stranded β-sheet and the following helix bundle (FIG. 1B). The understanding of the overall topology, localization of the conserved residues prompted an investigation on understanding the enzymatic function of the NiRAN domain. For this, two different approaches were considered with the Protein data bank as the target database. The first approach utilized the prediction of Hidden Markov Models(16) while the second approach predicted the presence of similar folds(17).Notably, both approaches predicted a variety of kinase and kinase like transferase (phosphotransferase molecules). A previous report had evidenced at the similarity of the

NiRAN domain to that of a known pseudokinase molecule SelO(5); however further characterization was not feasible due to absence of the NiRAN 3D structure. The inventors of the present invention compared the topologies of the NiRAN domain with that of the canonical kinase fold. Similar to the canonical kinase fold, the NiRAN domain comprises of an antiparallel β-sheet flanked by alpha helices. The canonical kinase domain exhibits a 5 stranded antiparallel β-sheet, which is flanked by two helices running parallel and one helix running perpendicular to the β-sheet (FIG. 1C, middle panel) (18, 19). However, the NiRAN domain shows a 4 stranded antiparallel β-sheet (S1-S4) flanked by one parallel (H2) and two perpendicular helices (H1 and H3) (FIG. IC, left panel). Further investigation into the available literature confirmed the presence of many non-canonical kinase folds, one of which presents a 4 stranded antiparallel β-sheet flanked by three parallel and one perpendicular helix (FIG. 1C, left panel) (20).Taken together, these results suggest that the NiRAN domain assumes a kinase like fold, possibly functioning either as a kinase or a phosphotransferase.

The earlier study with EAV-RdRp experimentally demonstrated the binding of GTP and UTP nucleotides to the NiRAN domain (8). A docking analysis of these nucleotides with the NiRAN domain was performed to delineate the probable active site. Kinase domains in general possess an active site between the antiparallel β-sheet and the subsequent helix bundle (18, 21, 22). Both GIP (FIG. 2A& A′) and UTP (FIG. 2 B & B′) docked well within the probable active site region with docking scores of -9.84 kcal/mol and -6.59 kcal/mol, respectively. Besides, end-point binding free energy calculation with MM/GBSA suggest a strong interaction between the nucleotides and the residues in the probable active site (-15.77 kcal/mol and -17.67 kcal/mol for GTP and UTP, respectively). An overview of the active site pocket depicting the electrostatic surface potential indicates the localization of charged residues at the entry points to the site, while the interior of the pocket remains lined with largely non-polar residues (FIG. 2C).

1.2 Analysis of the kinase activity of NiRAN domain

In order to further explore the kinase like catalytic nature of NiRAN domain, three broad specificity kinase inhibitors-Sunitinib, Sorafenib and SU6656 were randomly selected and docked into the predicted active site of the NiRAN domain. All three kinase inhibitors show strong binding at the predicted active site as evident from the low free energy of binding (FIG. 3A, A′, B, B′, C and C′, Table 1). Interestingly, these inhibitors also demonstrate potential H-bond with residues lining the active site.

As mentioned earlier, this domain might be involved in GTP induced protein phosphorylation, thus enabling a primer independent RNA replication (G-capping) (8). Also, the domain may be involved in phosphorylation of UTP, thus functioning as a terminal nucleotidyltransferase. A wide range of viruses, both with DNA and RNA genomes are known to possess either multifunctional or dedicated proteins exhibiting the aforementioned activities. After careful examination, a list of 77 compounds with experimentally demonstrated inhibitory potential against members of Flaviviridae, Togaviridae; Human cytomegalovirus, Herpes simplex virus, and few gram negative bacterial species were selected for docking against the CoV-2-RdRp NiRAN domain active site (23-30).The five inhibitors with the best docking in scores in a decreasing order are- 65482,122108,135659024,4534, and23673624 (numbers represent the PubChem IDs). All these five inhibitors occupy varying regions within the active site pocket in a manner that their aromatic rings align with the uncharged/non-polar regions, while the charged moieties fit in the extremities of the active site pocket (FIG. 4A-E, Table 2).The molecular interactions of these inhibitors with the CoV-2-RdRp NiRAN domain active site are presented in FIG. 5A′-E′.

In order to determine any putative kinase like activity being harboured by the CoV-2 RdRp, the protein was overexpressed, purified (FIG. 5A & B) and its identity was confirmed by mass spectrometry (FIG. 5C). An ADP-Glo Kinase assay kit was utilized to determine the kinase activity of the CoV-2 RdRp. The efficiency of the assay in accurately determining a kinase activity was verified using a known kinase—human Akt2. The enzyme exhibited significant activity with a Km value of˜300 μM for ATP (FIG. 6A), which is very similar to the Kₘ values (˜ 350 μM) determined in previously reported studies. Also, Bovine Serum Albumin, a protein that binds ATP exhibited negligible activity (FIG. 6A), further verifying the specificity and selectivity of the protocol. Notably, incubation of the CoV-2 RdRp with varying concentrations of ATP exhibited significant activity akin to that of the human Akt2, with a Kₘ of˜500 μM (FIG. 5A).

1.3 Confirming kinase activity of RdRp

To further ascertain the possible kinase like activity of CoV-2 RdRp, both CoV-2 RdRp and human Akt2 were incubated with excess of ATP and treated with 500 nM of the each of the three kinase inhibitors- Sorafenib, Sunitinib and SU6656. Interestingly, all the three kinases inhibitors significantly abrogated the kinase like activity of CoV-2 RdRp (FIG. 6B). For human Akt2, Sorafenib and SU6656 significantly inhibited its kinase activity, while Sunitinib treatment demonstrated a mild inhibition (FIG. 6B). The inventors were unable to procure the compound 23673624(6-MADTP) commercially, thus the four remaining compounds-135659024(m7GTP), 122108(Ribavirin 5′-triphosphate), 65482(Sinefungin) and 4534(Nordihydroguaiaretic acid) were used. The first three compounds were observed to have very little effects on the kinase like activity of CoV-2 RdRp at a concentration of 500 nM (FIG. 6C). However, these compounds which are nucleoside analogs/derivatives exhibited conspicuous inhibitions at a concentration of 1000 nM (FIG. 6C). The fourth compound-4534, a catechol derivative failed to inhibit the CoV-2 RdRp kinase like activity at any of the two concentrations (FIG. 6C).

2. Ex-vivo studies and results

To assess the effect of the kinase inhibitors in an ex-vivo infection, epithelial cells infected with SARS-CoV-2 were treated with concentrations of these compounds ranging from 1-50 μM. The approved anti-COVID-19 drug, Remdesivir was used as the control. The anti-kinase drug Sorafenib exhibited significant reduction in the infection, similar to that of Remdesivir (FIG. 6D). Further, a combination of Sorafenib and Remdesivir in variable concentration has synergistic effect on the controlling the infection by clearing the virus completely (FIG. 7).

TABLE 1
Docking analysis of kinase inhibitors at
the proposed active site of NiRAN domain.

| | | | Docking | Binding | |
|---|---|---|---|---|---|
| | | PubChem | Score | Energy | H-bond |
| No. | Name | ID | (Kcal/Mol) | (ΔG bind) | Interactions |
| 1 | Sunitinib | 5329102 | −3.50 | −28.07 | Asp36, |
| | | | | | Asp218 |
| | | | | | and Asp221 |
| 2 | Sorafenib | 216239 | −3.06 | −36.06 | Asp36 |
| 3 | SU6656 | 5312137 | −2.61 | −25.07 | Lys73 |

TABLE 2
Docking analysis of best 5 proposed inhibitors
at the proposed active site of NiRAN domain.

| | | Docking | Binding | | |
|---|---|---|---|---|---|
| | PubChem | Score | Energy | H-bond | Other |
| No. | ID | (Kcal/Mol) | (ΔG bind) | Interactions | Interactions |
| 1 | 65482 | −10.35 | −25.38 | Asp36, | Phe35 |
| | (Sinefungin) | | | Asn52, | (Pi-Pi), |
| | | | | Lys73, | Phe48 |
| | | | | Asp218, | (Pi-Pi), |
| | | | | Asp221 | Glu83 |
| | | | | | (Salt Bridge) |
| 2 | 122108 | −7.42 | −10.03 | Lys73, | Lys73 |
| | (Ribavirin 5′- | | | Asp36, | (Salt Bridge) |
| | triphosphate) | | | Asn52, | |
| | | | | Asp218, | |
| | | | | Asp221 | |
| 3 | 135659024 | −6.94 | −24.33 | Asn79, | Lys73 |
| | (m7GTP) | | | Thr76, | (Salt bridge), |
| | | | | Arg74, | Asp218 |
| | | | | Asn52, | (Salt bridge) |
| | | | | Val204, | |
| | | | | Asn209, | |
| 4 | 4534 | −6.84 | −32.07 | Lys73, | NA |
| | (Nordihydroguaiaretic | | | Asn52, | |
| | acid) | | | Asp218 | |
| 5 | 23673624 | −6.76 | −34.18 | Arg33, | Lys73 |
| | (6-MADTP) | | | Thr51, | (Salt bridge), |
| | | | | Asn52, | Arg116 |
| | | | | | (Salt Bridge) |

Using computational docking and simulation, the present invention predicts possible inhibitor compounds against the NiRAN domain and provides compositions and methods employing use of such inhibitors for augmenting anti-viral action against various viral pathogens possessing NiRAN domain.

The compound Sunitinib is an inhibitor of the tyrosine family kinases (34); Sorafenib inhibits activities of both serine/threonine and tyrosine family kinases (35, 36); while SU6656 inhibits the Src family kinases (36, 37). While Sorafenib and Sunitinib are approved for medical use in cases of renal, hepatocellular and gastro-intestinal cancers, the compound SU6656 is an experimental molecule used to study the role of Src kinases in cell cycle (38, 39).

In addition to the kinase inhibitors, the compound 65482 (Sinefungin), is broad specificity microbial nucleotidyitransferase inhibitor, and is known to inhibit RNA replication in flaviviruses and herpes viruses (40, 41). The compound 122108 (Ribavirin 5′-triphosphate), inhibits the formation of g-capping of RNA in a wide range of viruses, such as Dengue virus, Hantaan virus and Hepatitis C virus (11, 42, 43). Officially known as m7GTP, the compound with the PubChem Id 135659024, interferes with the RNA/DNA g-capping activity in many viral pathogens such Rift valley fever virus, Influenza virus, Zika virus and Dengue virus, to name a few (44-46).

The results suggest that the NiRAN domain of the SARS-CoV-2 RdRp possesses a kinase/phosphotransferase like enzymatic activity which is inhibited significantly by anti-cancer drugs and various anti-microbials targeting the NiRAN active site. In addition, the anti-kinase drug Sorafenib significantly reduces viral load in an ex-vivo SARS-CoV-2 infection in cell line infection.

We claim:

1. A method of inhibiting viral multiplication and treating a viral disease comprising:
co-administering, to a subject, a therapeutically effective amount of one or more anti-viral drugs and a therapeutically effective amount of at least one or more anti-kinase drugs,
wherein the method inhibits kinase activity of a NiRAN domain of RNA dependent RNA polymerase of a target virus.

2. The method according to claim 1, wherein the one or more anti-viral drugs is selected from Sinefungin, Ribavirin 5′-triphosphate, m7GTP, Nordihydroguaiaretic acid, Remdesivir and 6-MADTP.

3. The method according to claim 1, wherein the one or more anti-kinase drugs is selected from Sunitinib, Sorafenib and SU6656.

4. The method according to claim 1, wherein the one or more anti-viral drugs and the one or more anti-kinase drugs are administered sequentially or simultaneously in combination.

5. The method according to claim 1, wherein the target virus is selected from a family of nidoviruses and any other virus having the NiRAN domain.

6. The method according to claim 1, wherein the target virus is SARS-CoV-2 and the viral disease is COVID-19.

7. The method according to claim 1, wherein the subject is a mammal.

8. The method according to claim 7, wherein the mammal is a human.

9. A composition comprising:
a therapeutically effective amount of at least one or more anti-viral drugs selected from Sinefungin, Ribavirin 5′-triphosphate, m7GTP, Nordihydroguaiaretic acid, Remdesivir and 6-MADTP; and
a therapeutically effective amount of at least one or more anti-kinase drugs selected from Sunitinib, Sorafenib and SU6656,
wherein the one or more anti-kinase drugs augments an antiviral efficacy of the one or more anti-viral drugs.

10. A system comprising:
a therapeutically effective amount of at least one or more anti-viral drugs including Sinefungin, Ribavirin 5′-triphosphate, m7GTP, Nordihydroguaiaretic acid, Remdesivir and 6-MADTP;
a therapeutically effective amount of at least one or more anti-kinase drugs including Sunitinib, Sorafenib and SU6656; and
a target virus.

11. The system according to claim 10, wherein the target vines is selected from a family of nidoviruses and any other virus having a NiRAN domain.

12. The system according to claim 10, wherein the target virus is SARS-CoV-2.

13. The system of claim 10, wherein the target virus resides in a mammal.

14. The system according to claim 13, wherein the mammal is a human.
